(19) United States
(12) Patent Application Publication
(10) Pub. No.: US 2021/0128783 A1
(43) Pub. Date: May 6, 2021

(54) LAMINATED SKIN MIMETIC

(71) Applicant: L'Oreal, Paris (FR)

(72) Inventor: Michael J. Kearney, Snohomish, WA (US)

(73) Assignee: L'Oreal, Paris (FR)

(21) Appl. No.: 17/061,245

(22) Filed: Oct. 1, 2020

Related U.S. Application Data
(60) Provisional application No. 62/928,070, filed on Oct. 30, 2019.

(51) Int. Cl. A61L 27/36; A61L 27/52; A61L 27/24; A61L 27/22; A61L 27/20; A61L 27/16

(52) U.S. Cl. CPC A61L 27/362; A61L 27/52; A61L 27/24; A61L 27/16; A61L 27/227; A61L 27/3687; A61L 27/20; A61L 27/222

(57) ABSTRACT

The present disclosure features a skin mimetic, including a base layer including a hydrogel; and a top layer including a synthetic stratum corneum. The skin mimetic mimics a skin portion of a human subject, such as a facial skin portion. In an embodiment, the skin mimetic can serve as a mimic for a cheek skin portion, for example, for use in the testing of cosmetic products, therapeutic agents, moisturizers, shaving products, other skin care products, and the like.

### CROSS-REFERENCE TO RELATED APPLICATION

This application claims the benefit of Provisional Application No. 62/928,070, filed Oct. 30, 2019, the entire disclosure of which is incorporated herein by reference in its entirety.

### SUMMARY

In one aspect, the present disclosure features a skin mimetic, including a base layer including a hydrogel; and a top layer including a synthetic stratum corneum.

In another aspect, the present disclosure features a method of making a human skin model, including providing a base layer including a hydrogel including water; adhering a top layer to the base layer; and wherein the top layer is hydrated with the water from the base layer.

The summary above is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic illustration of an embodiment of a skin mimetic of the present disclosure.

FIG. 2 is a photograph of an embodiment of a skin mimetic of the present disclosure.

FIG. 3A shows comparative photographs of a bare skin portion with an embodiment of a skin mimetic of the present disclosure.

FIG. 3B shows a series of bar graphs comparing a bare skin portion with an embodiment of a skin mimetic of the present disclosure.

FIG. 4 is a photograph of an embodiment of a skin mimetic of the present disclosure.

### DETAILED DESCRIPTION

Human skin is often categorized into thick skin, with a thickness of 400-600 μm, and thin skin, with a thickness of 75-150 μm. It is formed by a dermal layer, i.e. the dermis, formed of a network of collagen, elastin, fibronectin, and fibroblasts, an epidermal layer, of keratinocytes, melanocytes, Langerhans cells, Merkel cells, and a dermal-epidermal junction (basement membrane) between the epidermis and dermis. The skin can possess a stiffness of 185 N/m to 300 N/m, depending on area of the skin, hydration levels, and age.

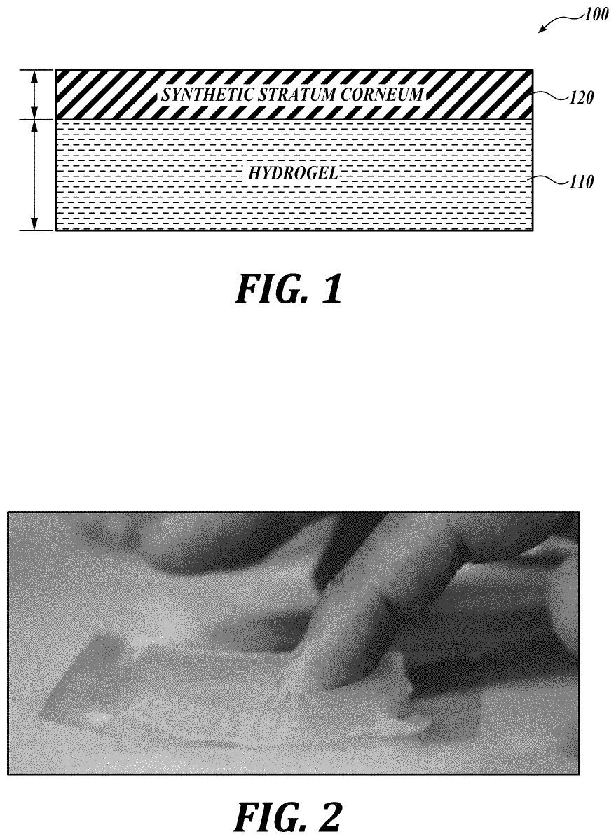

Referring to FIG. 1, in an embodiment, the present disclosure features a skin mimetic 100, including a base layer 110 including a hydrogel; and a top layer 120 including a synthetic stratum corneum. In an embodiment, the top layer is thinner than the base layer. For example, the base layer can have a thickness of about 5 mm, and the top layer can have a thickness of about 0.1 mm. The skin mimetic mimics a skin portion of a human subject, such as a facial skin portion. In an embodiment, the skin mimetic can serve as a mimic for a cheek skin portion, for example, for use in the testing of cosmetic products, therapeutic agents, moisturizers, shaving products, other skin care products, and the like.

In an embodiment, the skin mimetic has an adhesive layer between the base layer and the top layer, where the adhesive layer is different from the base and the top layer. In an embodiment, the base layer itself has adhesive properties, and the resulting skin mimetic can be devoid of an additional adhesive layer between the base layer and the top layer.

In an embodiment, the top layer includes a synthetic proteinaceous material, which includes a modified natural proteinaceous material. In an embodiment, the top layer includes a collagen, a gelatin, a glycosaminoglycan, a chondroitin sulfate, a silicone, or any combination thereof.

In an embodiment, the top layer includes Vitro-Skin®, Vitro-Corneum®, both available from Florida Skincare Testing, IMS Division; and Tegaderm™, available from 3M. In some embodiments, the top layer includes Test Skin™ II from Organogenesis Inc., Canton, Mass.; Skinethic® from Skinethic Tissue Culture Laboratories, Nice, France; EpiDerm™ simulated human skin from MatTek Corporation, Ashland, Mass.; a medical grade collagen film; a collagen in a sausage casing; cellulose film, a custom prepared chamois available from Acme Sponge and Chamois Company, Tarpon Springs, Fla.; a cultured or bioengineered skin substrate; and/or similar materials.

Without wishing to be bound by theory, it is believed that Vitro-Skin® is an advanced testing substrate that effectively mimics the surface properties of human skin. It is formulated to have topography, pH, critical surface tension, chemical reactivity and ionic strength that are similar to human skin.

Without wishing to be bound by theory, it is believed that Vitro-Corneum® is a collagen-based substrate with properties similar to human stratum corneum, and is designed to simulate the thickness, visco-elasticity, chemical reactivity, and surface properties of human stratum corneum; i.e., the outer layer of epidermis of primarily dead skin cells. It is believed that Vitro-Corneum® is made from a single layer of a gelatin and protein-based material. It can be used to demonstrate the hydration and moisturization properties of personal care products and raw materials in vitro. Vitro-Corneum® provides an experimentally simple approach to evaluating the benefits of prototype formulations and new raw materials—without the experimental difficulties of working with and separating human epidermis. Like human stratum corneum, the visco-elastic properties of Vitro-Corneum® change dramatically with changes in hydration.

Without wishing to be bound by theory, it is believed that the EpiDerm™ bioengineered human skin incorporates stratified epidermal layers, including a functional stratum corneum.

Another suitable simulated skin substrate is collagen. An exemplary collagen is available from Naturin GmbH, Weinhein, Germany, under the designation of COFF12224. COFF12224 is a collagen film having a basis weight of about 28 g/m². Another exemplary collagen film is available from Devro, Inc, Geneva, Ill., under the designation of Cutisin™.

In an embodiment, the base layer includes a hydrogel, which includes a crosslinked polymer, a crosslinked copolymer, or a crosslinked multipolymer interpenetrating network. As used herein, a hydrogel refers to a network of polymer chains that are hydrophilic, where a three-dimensional solid results from the hydrophilic polymer chains held together by crosslinks. The structural integrity of the hydrogel network does not dissolve from a high water concentration. In an embodiment, the hydrogel includes 90% or more (e.g., 92% or more, 95% or more, or 97% or more) of water. In an embodiment, the hydrogel includes a crosslinked polymer including a polysiloxane, a polyacrylate, a polyacrylamide, a polyurethane, a polyvinyl alcohol, a polysaccharide, a polyamino acid, copolymers thereof, and any combination thereof. In an embodiment, the skin mimetic is adapted to match the appearance and behavior (e.g., elasticity and/or permeability) of a human skin portion of a predetermined group.

In an embodiment, the hydrogel is an ultraviolet (UV) light- or heat-crosslinked hydrogel. When the top layer is adhered to the base layer, the water contained in the base layer hydrates the top layer. In an embodiment, the hydrogel is one available from Katecho, Inc. (Des Moines, Iowa), such as KM30B and/or KM10C, having the properties listed in Example 1, below. In an embodiment, the hydrogel includes a mixture of an hydroxyl-terminated silicone polymer and methyl triethoxy silane which moisture-cures into a flexible, tough layer that has adherent properties; polyacrylate or polymethacrylate esters or their copolymers such as an acrylic rubber latex formed from an ethyl acrylate-acrylic acid copolymer; and/or polyurethanes such as a reaction product of excess toluene diisocyanate with a mixture of diols and triols to give a reactive, moisture-curing prepolymer capable of forming an elastomeric layer.

In an embodiment, the skin mimetic is capable of being sterilized, i.e., of being subjected to physical or chemical treatment that kills bacteria and bacterial spores on its surface, without affecting the physical characteristics of the skin mimetic. Suitable sterilization techniques include dry heat, exposure to ethylene oxide, irradiation, immersion in glutaraldehyde solution, and the like.

In an embodiment, the skin mimetic further includes a pigment incorporated into and/or coated onto the top layer, the base layer, or both the top layer and the base layer. The skin mimetic can be adapted to match the color of a predetermined Fitzpatrick skin type. In an embodiment, pigments include bismuth oxychloride-based pigments, mineral pigments, borosilicate-based interference pigments, synthetic mica pigments, and/or mica-based pigments. In an embodiment, the skin mimetic is permeable to a number of agents, such as a therapeutic agent, moisture, a hydrating agent, a lipid, and the like. In an embodiment, when viewed microscopically, the skin mimetic surface includes a number of pores. In an embodiment, when viewed microscopically, the skin mimetic surface includes a number of hair follicle-like structures.

In an embodiment, the skin mimetic has a skin property parameter (Rn, where n=0-9, see Table 1) value that differs by less than 10% from a corresponding skin property parameter (Rn, where n=0-9)) value for a human skin of a predetermined age group. For example, in an embodiment, the skin mimetic has an overall elasticity of the skin including creep and creep recovery value (R2) value that differs by less than 10% (e.g., less than 8%, or less than 5%) from an R2 value for a human skin of a predetermined age group (e.g., 18 to 25 years old, 25 to 40 years old, 40 to 50 years old, 50 to 60 years old, or 60 years and older). In an embodiment, the skin mimetic has a ratio of elastic recovery to the total deformation (R7) value that differs by less than 10% from an R7 value for a human skin of a predetermined age group.

In an embodiment, the skin mimetic has properties that are measured using a Cutometer® Dual MPA 580 instrument, available from Courage+Khazaka Electronic GmbH. The measuring principle of the Cutometer® is based on suction, where Negative pressure created by a vacuum pump within the device draws the skin into an aperture of a probe. Inside the probe, the penetration depth is determined by a non-contact optical measuring system consisting of a light source and a light receptor, as well as two prisms facing each other, which project the light from transmitter to receptor. The light intensity varies due to the penetration depth of the skin. The resistance of the skin to be sucked up by negative pressure (firmness) and its ability to re-turn into its original position (elasticity) are dis-played as curves in real time. From these curves a variety of interesting measurement parameters can be calculated related to elastic and visco-elastic properties of skin surface and skin aging.

In a skin sample from a human subject, the typical shape of a curve of human skin is based on the different forces of elastin and collagen in the skin. Elastin is responsible for the flexibility of the skin whereas collagen's main task is to keep the skin in shape. The first straight part of the curve is shaped by the portion of elastin in the skin as it easy to displace and very flexible. When skin starts to “creep” inside the probe, the collagen has taken over. It is stronger and resists the mechanical force better. Immediately after the pressure of the device has ceased, the collagen let go. Therefore, in young skin with fresh collagen, the skin instantly returns better to its original position than in more aged skin. In the end, eventually the elastin sees to the complete recovery of the skin.

TABLE 1
Skin property parameters obtainable from Cutometer ® measurements
and calculations

| Parameter | Representation | Equivalent |
|---|---|---|
| R0 | The final distension of the first curve | Uf |
| R1 | The ability to return to the original state | Uf − Ua |
| R2 | The overall elasticity of the skin, including | Ua/Uf |
| | creep and creep recovery | |
| R3 | The last maximum highest point of the last curve | Last Uf |
| R4 | The last minimal lowest point of the last curve | Last R |
| R5 | The net elasticity | Ur/Ue |
| R6 | The ratio of viscoelastic to elastic extension, | Uv/Ue |
| | also called the viscoelastic ratio | |
| R7 | The ratio of elastic recovery to the total | Ur/Uf |
| | deformation | |
| R8 | The Ua of the first curve | First Ua |
| R9 | The residual deformation at the end of the | R3 − R0 |
| | measuring cycle | |

Where, in Table 1, Uf is the final distension at the end of first vacuum period; Ua is the difference between the maximum deformation of the first vacuum period and the deformation after 1 second of normal pressure; Ur is the immediate relaxation within the first 0.1 second after the end of the first vacuum period; Ue is the immediate distension of the skin within the first 0.1 second of the first vacuum period; and Uv is the difference between the deformation after 0.1 second and the maximal deformation of the first vacuum period.

In an embodiment, the skin mimetic has a relatively large surface area. For example, in an embodiment, the skin mimetic has a ratio of a thickness of the top layer to a thickness of base layer is from 1:30 to 1:100 (e.g., 1:30 to 1:80; 1:50-1:100, 1:50 to 1:80, or 1:50).

The skin mimetic can be made according to any method as known to a person of ordinary skill in the art. In an embodiment, the skin mimetic, which can serve as a human skin model, can be made by providing a base layer including a hydrogel including water; and adhering a top layer to the base layer. Once adhered to the base layer, the top layer is hydrated with the water from the base layer. When the top layer is hydrated, the skin mimetic is supple and can provide a skin model suitable for a variety uses, such for cosmetic testing, medical testing, artificial dressing, skin replacement, and the like. The top and base layers can be made according to methods known to a person of ordinary skill in the art.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means+/−5%. As used herein, a recited ranges includes the end points, such that from 0.5 percent to 99.5 percent includes both 0.5 percent and 99.5 percent.

As used herein, the singular forms “a,” “an” and “the” include plural referents unless the context clearly dictates otherwise. Thus, for example, reference to “a polypeptide” includes a mixture of two or more polypeptides and the like.

As used herein “animal” means a multicellular, eukaryotic organism classified in the kingdom Animalia or Metazoa. The term includes, but is not limited to, mammals. Nonlimiting examples include rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals.

As used herein the term “patient” is understood to include an animal, especially a mammal, and more especially a human that is receiving or intended to receive treatment.

As used herein, “substantially” means largely if not wholly that which is specified but so close that the difference is insignificant, and such differences do not influence the functional properties of the term beyond the normal tolerances permitted by one of skill in the art. In some embodiments, “substantially” means that the differences do not vary by more than 10% or less.

As used herein, the terms “simulated,” “artificial,” “synthetic” and like terms are used interchangeably to indicate manufactured materials or objects, and in the case of substrates, dissected or bio-engineered skin samples, unless otherwise indicated.

The following examples are provided to illustrate, not limit, the disclosure.

### EXAMPLES

### Example 1: Preparation of Skim Mimetic Samples and Comparison of Differences from Bare Skin

A skin mimetic having two samples was made. The top layer (representative of the stratum corneum) was a gelatin and protein-based synthetic skin substrate, specifically Vitro-Corneum®. The bottom layer (representative of the dermis) is formed of an adhesive hydrogel (e.g., a hydrogel formed of acrylate-acrylamide copolymer), obtained from Katecho Inc. (Des Moines, Iowa) as hydrogel KM30B or KM 10C. Varying the amount of hydrogel firmness can mimic certain age demographics. The two layers work together to complement one another. The hydrogel supplies needed moisture to the Vitro-Corneum® (VC) and the VC protects the hydrogel from drying up. The top layer had a thickness of 0.1 mm and the bottom layer had a thickness of 4.9 mm. The properties of KM30B and KM10C are listed in Tables 2 and 3, below.

TABLE 2
Material properties of Katecho KM30B hydrogel

| Property/Test Method | Values |
|---|---|
| Color | Transparent blue |
| Volume resistivity | 294 Ohm-in |
| Stainless steel adhesion | 240 grams per inch width |
| Top liner adhesion | 95 grams per inch width |
| Bottom liner adhesion | 101 grams per inch width |
| Hydrogel process pH | 4.65 |
| Thickness of hydrogel | 40 mils (±5 mils) |
| Peak tack | 75 grams |
| Leg (stretch) | 3.0 mm |
| Work of adhesion | 232 gram sec |

KM30B has a soft feeling, is repositionable, and has good dry-out resistance.

TABLE 3
Katecho KM10C hydrogel properties

| Property/Test Method | Values |
|---|---|
| Color | Transparent blue |
| Volume resistivity | 100 Ohm-in |
| Stainless steel adhesion | 485 grams per inch width |
| Top liner adhesion | 81 grams per inch width |
| Bottom liner adhesion | 120 grams per inch width |
| Hydrogel process pH | 6.0 |
| Thickness of hydrogel | 40 mils (±5 mils) |
| Peak tack | 56 grams |
| Leg (stretch) | 4.6 mm |
| Work of adhesion | 272 gram sec |

KM10C has high water content, a salt-free formulation, and is aluminum substrate compatible.

The VC was hydrated for 4-6 hours prior to application to the hydrogel layer. Hydration can be achieved in a hydration chamber in a container that suspends VC above the hydration solution. VC was cut to the appropriate size needed prior to being placed in the hydration chamber. The hydration solution was a mixture of water and glycerin. In the hydration chamber 52 grams of glycerin and 298 grams of water were combined to form a solution having 85% water and 15% glycerin. The solution was not in direct contact with the VC.

After hydration, the precut VC layer was removed from the hydration chamber, and applied atop the hydrogel layer. Application was immediate and with caution to avoid drying and air bubbles forming between the two layers. The KM 10C and KM 30B hydrogel have very strong adhesion properties. Applying the VC to encompass all areas of the hydrogel ensures sufficient hydration of the VC to stay supple for several days.

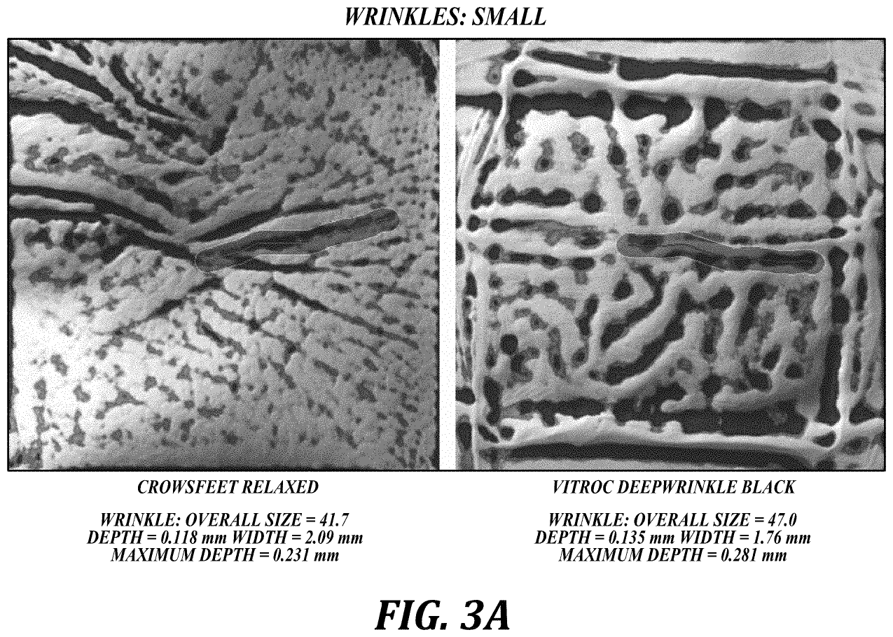

FIG. 2 is a photograph of an example of a newly made skin mimetic with a ¼ inch thick KM30B hydrogel and a VC layer on top of the hydrogel. Pressure was applied on top of skim mimetic to demonstrate the pliability and softness. The skin mimetic bounced back, or retained its original shape immediately after applying pressure.

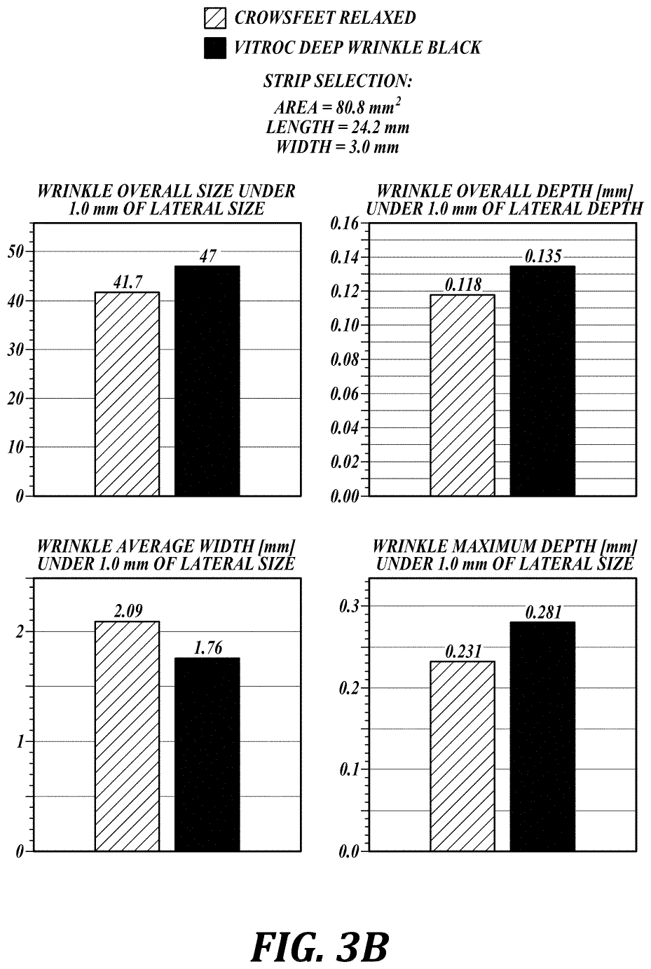

FIG. 3A shows a photographic comparison of the skin mimetic compared to a human skin portion. The photographs were taken with an Antera 3D camera, which has the capability to measure fine lines and wrinkles. The left-hand photograph is an image of a human skin sample corresponding to crow's feet wrinkles at the corner of an eye. The right-hand image shows a skin mimetic sample of the present Example, having wrinkles. The wrinkles in both images are similar. The graphs in FIG. 3B compares the overall wrinkle size, average wrinkle depth, average wrinkle width, and maximum wrinkle depth of the crow's feet wrinkles (left bar in red) and the wrinkles in the skin mimetic (right bar in blue), showing a close correlation.

Table 4 below shows the comparison of Cutometer® data collected from a skin mimetic of the present Example, made with VC and either KM10C or KM30B, with bare skin cheek measurements from 10 human subjects aged 18-65. R0-R8 measurements were obtained using the Cutometer® instrument instructions and as described above in the Detailed Description The Cutometer® measurements and calculations from bare skin were performed at 3 different time points. The data from the skin mimetic were collected at 4 different points in a square pattern on a rectangular skin mimetic sample. The R0-R8 differences of the skin mimetic samples vs. bare skin are shown in Table 4, below.

TABLE 4
Comparison of properties of skin mimetic samples made
with KM10C or KM30B with an average of skin properties
of a panel of subjects between 18 and 65 years old.

| Bare | Bare | Bare | Bare | Bare | Bare | Bare | Bare | Bare |
|---|---|---|---|---|---|---|---|---|
| skin vs | skin vs | skin vs | skin vs | skin vs | skin vs | skin vs | skin vs | skin vs |
| KM10C | KM10C | KM10C | KM10C | KM10C | KM10C | KM10C | KM10C | KM10C |
| R0 | R1 | R2 | R3 | R4 | R5 | R6 | R7 | R8 |
| −0.02 | 0.09 | −0.23 | −0.02 | 0.09 | −0.24 | −0.01 | −0.18 | −0.11 |
| Bare | Bare | Bare | Bare | Bare | Bare | Bare | Bare | Bare |
| skin vs | skin vs | skin vs | skin vs | skin vs | skin vs | skin vs | skin vs | skin vs |
| KM30B | KM30B | KM30B | KM30B | KM30B | KM30B | KM30B | KM30B | KM30B |
| R0 | R1 | R2 | R3 | R4 | R5 | R6 | R7 | R8 |
| 0.17 | 0.08 | −0.10 | 0.17 | 0.08 | −0.21 | -1.33 | 0.00 | 0.09 |

### Example 2. Preparation of a Skin Mimetic with Tegaderm™

Tegaderm™ film is a transparent thin film backing with a non-latex, hypoallergenic adhesive. The film is breathable, allowing good oxygen and moisture vapor exchange. It is waterproof and impermeable to liquids, bacteria, and viruses. The dressing provides a viral barrier from viruses 27 nm in diameter or larger while the dressing remains intact without leakage. The dressing can conform to a skin surface to which it is applied.

A skin mimetic with Tegaderm™ as the top layer and a hydrogel (KM10C or KM30B) was made in an analogous manner as the preparation method in Example 1, with the exception that Tegaderm™ was used instead of VC. Tegaderm™ had an adhesive side that was attached to the hydrogel surface.

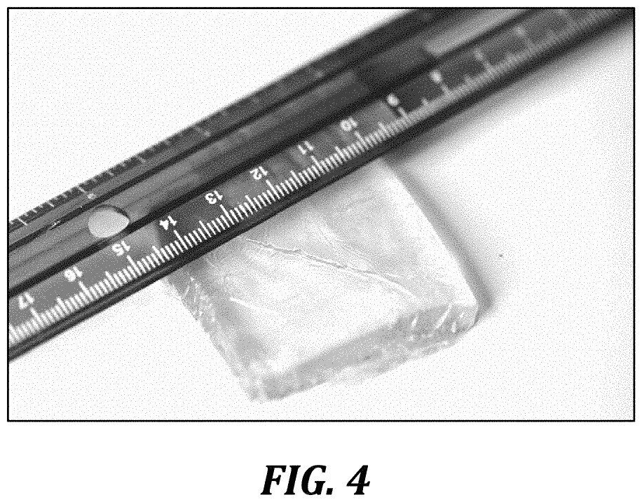

FIG. 4 shows a photograph of a skin mimetic made with a hydrogel and Tegaderm™, showing wrinkles on the skin mimetic. The skin mimetic can be subjected to Cutometer® analysis as described in the Detailed Description and in Example 1, above.

Although the subject matter for which protection is sought is defined in the appended claims, other illustrative, non-exclusive examples of subject matter according to the present disclosure are described in the following enumerated paragraphs:

A.1. A skin mimetic, comprising

a base layer comprising a hydrogel; and

a top layer comprising a synthetic stratum corneum.

A.2. The skin mimetic of A.1, further comprising an adhesive layer between the base layer and the top layer.

A.3. The skin mimetic of A.1 or A.2, wherein the base layer is an adhesive layer.

A.4. The skin mimetic of any one of A.1 to A.3, wherein the top layer comprises a collagen, a gelatin, a glycosaminoglycan, a chondroitin sulfate, a silicone, or any combination thereof.

A.5. The skin mimetic of any one of A.1 to A.4, wherein the hydrogel comprises a crosslinked polymer, a crosslinked copolymer, or a crosslinked multipolymer interpenetrating network.

A.6. The skin mimetic of A.5, wherein the crosslinked polymer or copolymer is selected from a polysiloxane, a polyacrylate, a polyurethane, a polyvinyl alcohol, a polysaccharide, a polyamino acid, and any combination thereof.

A.7. The skin mimetic of any one of A.1 to A.6, wherein the top layer comprises a synthetic protein.

A.8. The skin mimetic of any one of A.1 to A.7, adapted to match the appearance and elasticity of a human skin composition of predetermined age group.

A.9. The skin mimetic of any one of A.1 to A.8, further comprising a pigment in the top layer, the base layer, or both the top layer and the base layer.

A.10. The skin mimetic of A.9, adapted to match the color of a predetermined Fitzpatrick skin type.

A.11. The skin mimetic of any one of A.1 to A.10, wherein the skin mimetic is permeable.

A.12. The skin mimetic of any one of A.1 to A.10, wherein the top layer is selected from Vitro-Corneum® and Tegaderm™.

A.13. The skin mimetic of any one of A.1 to A.12, wherein the base layer is an ultraviolet light- or heat-crosslinked hydrogel.

A.14. The skin mimetic of any one of A.1 to A.13, wherein the base layer is configured to hydrate the top layer.

A.15. The skin mimetic of any one of A.1 to A.14, wherein the skin mimetic has a skin property parameter (Rn, where n=0-9) value that differs by less than 10% from a skin property parameter (Rn, where n=0-9)) value for a human skin of a predetermined age group.

A.16. The skin mimetic of any one of A.1 to A.15, wherein the skin mimetic has an overall elasticity of the skin including creep and creep recovery value (R2) value that differs by less than 10% from an R2 value for a human skin of a predetermined age group.

A.17. The skin mimetic of any one of A.1 to A.15, wherein the skin mimetic has a ratio of elastic recovery to the total deformation (R7) value that differs by less than 10% from an R7 value for a human skin of a predetermined age group.

A.18. The skin mimetic of any one of A.1 to A.17, wherein a ratio of a thickness of the top layer to a thickness of base layer is from 1:30 to 1:100 (e.g., 1:50).

A.19. A method of making a skin mimetic of any one of A.1 to A.18, comprising: providing a base layer comprising a hydrogel comprising water;

adhering a top layer to the base layer; and

wherein the top layer is hydrated with the water from the base layer.

A.20. A method of making a human skin model, comprising:

providing a base layer comprising a hydrogel comprising water;

adhering a top layer to the base layer; and

wherein the top layer is hydrated with the water from the base layer.

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A skin mimetic, comprising:
a base layer comprising a hydrogel; and
a top layer comprising a synthetic stratum corneum.

2. The skin mimetic of claim 1, further comprising an adhesive layer between the base layer and the top layer.

3. The skin mimetic of claim 1, wherein the base layer is an adhesive layer.

4. The skin mimetic of claim 1, wherein the top layer comprises a collagen, a gelatin, a glycosaminoglycan, a chondroitin sulfate, a silicone, or any combination thereof.

5. The skin mimetic of claim 1, wherein the hydrogel comprises a crosslinked polymer, a crosslinked copolymer, or a crosslinked multipolymer interpenetrating network.

6. The skin mimetic of claim 5, wherein the crosslinked polymer or copolymer is selected from a polysiloxane, a polyacrylate, a polyurethane, a polyvinyl alcohol, a polysaccharide, a polyamino acid, and any combination thereof.

7. The skin mimetic of claim 1, wherein the top layer comprises a synthetic protein.

8. The skin mimetic of claim 1, adapted to match the appearance and elasticity of a human skin composition of predetermined age group.

9. The skin mimetic of claim 1, further comprising a pigment in the top layer, the base layer, or both the top layer and the base layer.

10. The skin mimetic of claim 9, adapted to match the color of a predetermined Fitzpatrick skin type.

11. The skin mimetic of claim 1, wherein the skin mimetic is permeable.

12. The skin mimetic of claim 1, wherein the top layer is selected from Vitro-Corneum® and Tegaderm™.

13. The skin mimetic of claim 1, wherein the base layer is an ultraviolet light- or heat-crosslinked hydrogel.

14. The skin mimetic of claim 1, wherein the base layer is configured to hydrate the top layer.

15. The skin mimetic of claim 1, wherein the skin mimetic has a skin property parameter (Rn, where n=0-9) value that differs by less than 10% from a skin property parameter (Rn, where n=0-9)) value for a human skin of a predetermined age group.

16. The skin mimetic of claim 1, wherein the skin mimetic has an overall elasticity of the skin including creep and creep recovery value (R2) value that differs by less than 10% from an R2 value for a human skin of a predetermined age group.

17. The skin mimetic of claim 1, wherein the skin mimetic has a ratio of elastic recovery to the total deformation (R7) value that differs by less than 10% from an R7 value for a human skin of a predetermined age group.

18. The skin mimetic of claim 1, wherein a ratio of a thickness of the top layer to a thickness of base layer is from 1:30 to 1:100 (e.g., 1:50).

19. A method of making a skin mimetic of claim 1, comprising:
providing a base layer comprising a hydrogel comprising water;
adhering a top layer to the base layer; and
wherein the top layer is hydrated with the water from the base layer.

20. A method of making a human skin model, comprising:
providing a base layer comprising a hydrogel comprising water;
adhering a top layer to the base layer; and
wherein the top layer is hydrated with the water from the base layer.
